(19) United States
(12) Patent Application Publication
(10) Pub. No.: US 2026/0264056 A1
(43) Pub. Date: Sep. 10, 2026

(54) METHOD FOR PREPARING PLATINUM-COATED HONEYCOMB CATALYST USED IN TOC WATER QUALITY MEASURING DEVICE

(71) Applicants: ANALTEC TMS CO., LTD., Daegu (KR); ATT CO., LTD., Cheonan-si (KR)

(72) Inventor: Jae Cheol KIM, Daegu (KR)

(21) Appl. No.: 18/849,246

(22) PCT Filed: Jun. 16, 2023
(86) PCT No.: PCT/KR2023/008406

§ 371 Date: Sep. 20, 2024

(30) Foreign Application Priority Data
Nov. 8, 2022 (KR) 10-2022-0148101

(51) Int. Cl. B01J 23/42; B01J 21/04; B01J 35/57; B01J 37/02; B01J 37/04; B01J 37/08

(52) U.S. Cl. CPC B01J 23/42; B01J 21/04; B01J 35/57; B01J 37/0225; B01J 37/04; B01J 37/08

(57) ABSTRACT

The present invention relates to a method for preparing a platinum-coated honeycomb catalyst used in a TOC water quality measuring device and, more specifically, to a method for preparing a platinum-coated honeycomb catalyst used in a TOC water quality measuring device, the platinum-coated honeycomb catalyst being used in a TOC water quality measuring device using a high-temperature thermal combustion method to maximize the efficiency of the water quality measuring device.

### TECHNICAL FIELD

The present invention relates to a method for preparing platinum-coated honeycomb catalyst used in a TOC water quality measuring device, and more specifically, to a method for preparing platinum-coated honeycomb catalyst used in a TOC water quality measuring device that can be used in a TOC water quality meter using a high-temperature thermal combustion method to maximize the efficiency of the water quality measuring device.

### BACKGROUND ART

Most countries, including Korea, manage water as one of the most important resources, and in particular, quality management of water including drinking water is basic management of the environment and the minimum basic requirement for human life. Countries around the world have set water quality standards to manage their water, but the water quality of the water circulating through the water system is becoming increasingly degraded due to the complex and diverse environmental pollution caused by industrialization.

As the need to use water quality analysis and measurement devices to increase the utilization value of water resources and prevent environmental pollution is increasing, the introduction of new measurement analysis methods and the acquisition of advanced measurement technology for water quality management and efficient water treatment facility operation are emerging as immediate challenges.

In general, among the representative indicators for measuring organic matter in water, biological oxygen demand (BOD), chemical oxygen demand (COD), and total organic carbon (TOC) are introduced.

As a device for continuous measurement of representative indicators of organic matter in water in an automated manner, various water quality continuous automatic meters are known, such as BOD meters, COD meters, and TOC meters.

However, since BOD meters use microorganisms as sensors, their performance can be degraded by toxic substances, and it is difficult to accurately measure organic matter in the presence of recalcitrant substances. COD meters have a large analytical error depending on the nature of the pollutant, and the measurement is affected by interfering substances such as chlorine.

Therefore, TOC meters (hereinafter referred to as TOC water quality measuring device) that analyze the degree of pollution by measuring the amount of organic carbon contained in water are mainly used in recent years, and TOC water quality measuring devices are used to monitor organic matter in boiler water, power plant cooling water, semiconductor processing, water purification plants, and sewage and wastewater treatment plants.

Prior art for such TOC measurements includes Korean Patent No. 10-1528126, “device for measuring total organic carbon and total nitrogen based on non-catalytic heat-combustion oxidization method.

This prior art includes the following components: a carrier gas pump that supplies carrier gas and air at a constant pressure for air bubbling at the rear end; a pump stage that includes a first pump sucking and supplying sample water to the next stage, a second pump sucking hydrochloric acid from a storage tank and supplying it to the next stage, and a third pump sucking the sample water that has been treated with acid and air bubbling from the pre-treatment sample storage port and supplying it the next stage; a 3-way mixing connector that mixes the sample provided by the first pump and the hydrochloric acid provided by the second pump in real time and provides sample water mixed with hydrochloric acid to the next stage; a pretreatment port that includes a first sample water inlet receiving sample water provided from the 3-way mixing connector, an air supply port receiving air supplied from the carrier gas pump, an air bubble forming unit discharging air bubbles internally by the positive pressure of the air received from the air supply port, and an outlet adding air bubbles to sample water mixed with hydrochloric acid to prepare homogeneous sample water and to supply it to the next stage; a pretreatment sample storage port that is provided with a second sample water inlet receiving sample water through the outlet of the pretreatment port and a second outlet discharging remaining sample water, and is installed with a sample suction straw sucking the acid and air-treated sample water provided by the third pump and providing it to a non-catalytic thermal combustion oxidation reactor for measurement in the next step; the non-catalytic thermal combustion oxidation reactor that oxidizes the sample supplied through the sample suction straw of the pretreatment sample storage port through thermal combustion; and a measuring unit that receives oxidized gas and measures the total organic carbon component or total nitrogen component.

However, conventional measuring devices have the problem of low measurement accuracy because suspended substances present in the sample coagulate and settle, acting as an interference factor in the measurement.

Further, if the inorganic carbon in the sample is not completely removed, the remaining inorganic carbon acts as an obstacle to the measurement of total organic carbon, so sufficient time for adding air bubbles to the sample must be provided, and therefore, there is a problem that the time required for total organic carbon measurement increases.

Further, the pretreatment port and the oxidation reactor are connected by a pipe, and the sample stored in the pretreatment port is moved to the oxidation reactor by a pump, and in this case, organic matter contained in the sample accumulates in the valve or pipe, causing the flow path to become blocked or narrowed, so that the desired flow rate is not transferred to reduce measurement accuracy, and organic residues accumulated in the valve or pipe contaminate the subsequent sample, which reduces measurement accuracy.

Prior art documents in the technical field to which the present invention belongs include Korean Patent No. 10-1528126, Korean Patent No. 10-1308496, etc.

### DISCLOSURE

### Technical Problem

The issues to be addressed by the present invention is to provide a method for preparing platinum-coated honeycomb catalyst used in TOC water quality measuring device in which the platinum catalyst is produced by including a platinum catalyst precursor and polyvinylpyrrolidone (PVP) to be used in a TOC water quality measuring device that uses high-temperature thermal combustion, thereby maximizing the efficiency of the water quality measuring device.

Further, the other issues to be addressed by the present invention is to provide a method for preparing platinum-coated honeycomb catalyst used in TOC water quality measuring device to manufacture a honeycomb catalyst with a platinum coating layer having excellent physical properties such as corrosion resistance, durability, and wear resistance.

Various problems to be solved by the present invention are not limited to the problems mentioned above, and other problems not mentioned are clearly understood by those skilled in the art from the description below.

### Technical Solution

In one embodiment of the technical idea of the present invention, a method for preparing platinum-coated honeycomb catalyst used in TOC water quality measuring device is disclosed.

The method for preparing platinum-coated honeycomb catalyst used in TOC water quality measuring device includes a honeycomb carrier preparation step (S100) of preparing a honeycomb carrier to be coated with a platinum catalyst; a honeycomb carrier cleaning step (S200) of cleaning the prepared honeycomb carrier with a cleaning solution; a platinum catalyst coating solution preparation step (S300) of preparing a platinum catalyst coating solution to be coated on the honeycomb carrier; a platinum catalyst coating solution applying step (S400) of coating the platinum catalyst coating solution on the honeycomb carrier; a drying step (S500) of drying the honeycomb carrier coated with the platinum catalyst coating solution; a heat treatment step (S600) of heat-treating the honeycomb carrier coated with the platinum catalyst coating solution; and a surface treatment step (S700) of adding a reaction gas to the heat-treated honeycomb carrier coated with the platinum catalyst coating solution to form a nitride layer thereon, thereby producing the platinum-coated honeycomb catalyst.

Details of other embodiments are included in the detailed description.

### Advantageous Effects

According to the method for preparing platinum-coated honeycomb catalyst used in TOC water quality measuring device according to various embodiments of the technical idea of the present invention, a platinum catalyst is prepared by including a platinum catalyst precursor and polyvinylpyrrolidone (PVP) to be used in a TOC water quality measuring device using high-temperature thermal combustion thereby maximizing the efficiency of the water quality measuring device.

Further, according to the method for preparing platinum-coated honeycomb catalyst used in TOC water quality measuring device according to various embodiments of the technical idea of the present invention, a honeycomb catalyst with a platinum coating layer having excellent physical properties such as corrosion resistance, durability, and wear resistance can be manufactured.

It is fully understood that various embodiments of the technical idea of the present invention may provide various effects not specifically mentioned.

### DESCRIPTION OF DRAWINGS

FIG. 1 is a flowchart illustrating a method for preparing platinum-coated honeycomb catalyst used in TOC water quality measuring device according to an embodiment of the technical idea of the present invention.

### MODE FOR INVENTION

Advantages and characteristics of the present invention, and methods for achieving them become clear with reference to the embodiments described later in detail. However, the present invention is not limited to the embodiments described herein and may be embodied in other forms. Rather, the embodiments introduced herein are provided so that the disclosed content is thorough and complete and the spirit of the present invention is sufficiently conveyed to those skilled in the art.

Terms used in this application are only used to describe specific embodiments and are not intended to limit the present invention. Singular expressions include plural expressions unless the context clearly dictates otherwise.

Unless defined otherwise, all terms used herein, including technical or scientific terms, have the same meaning as commonly understood by one of ordinary skill in the art to which the present invention belongs. Terms such as those defined in commonly used dictionaries should be interpreted as having a meaning consistent with the meaning in the context of the related art, and unless explicitly defined in this application, it is not to be construed in an ideal or overly formal sense.

Hereinafter, with reference to the attached drawings, a method for preparing platinum-coated honeycomb catalyst used in TOC water quality measuring device according to an embodiment of the technical idea of the present invention will be described in detail with preferred embodiments.

Meanwhile, the platinum-coated honeycomb catalyst according to an embodiment of the technical idea of the present invention is used in the reaction tube of a TOC water measuring device, and more specifically, the platinum-coated honeycomb catalyst is used in the reaction tube of a TOC water measuring device using a high-temperature thermal combustion method.

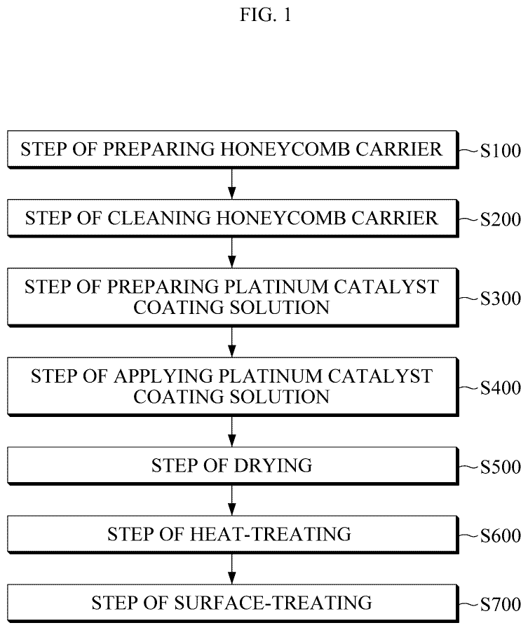

FIG. 1 is a flowchart illustrating a method for preparing platinum-coated honeycomb catalyst used in TOC water quality measuring device according to an embodiment of the technical idea of the present invention.

Referring to FIG. 1, the method for preparing platinum-coated honeycomb catalyst used in TOC water quality measuring device according to an embodiment of the technical idea of the present invention includes a honeycomb carrier preparation step (S100), a honeycomb carrier cleaning step (S200), a platinum catalyst coating solution preparation step (S300), a platinum catalyst coating solution applying step (S400), a drying step (S500), a heat treatment step (S600), and a surface treatment step (S700).

### 1. S100: Honeycomb Carrier Preparation Step

The honeycomb carrier preparation step (S100) is a step of preparing a honeycomb carrier to be coated with a platinum catalyst.

In the honeycomb carrier preparation step (S100), the honeycomb carrier can be manufactured using alumina or ceramic powder as a raw material, and for example, the honeycomb carrier may be prepared by using inorganic oxides such as activated alumina (γ-Al₂O₃) or ceramics that is fine and has good activity or a large specific surface area to increase the surface area of the carrier on which the platinum catalyst is supported.

In the present invention, the configuration of manufacturing a honeycomb carrier using the alumina or ceramic powder as a raw material is a known technology, so detailed description thereof is excluded for convenience of explanation and clarity of the technical idea of the present invention.

### 2. S200: Honeycomb Carrier Cleaning Step

The honeycomb carrier cleaning step (S200) is a step of cleaning the prepared honeycomb carrier with a cleaning solution to remove foreign substances attached to the surface of the honeycomb carrier and increase the surface area of the carrier.

In the honeycomb carrier cleaning step (S200), the prepared honeycomb carrier is immersed in a cleaning solution or cleaned with a cleaning solution to remove foreign substances attached to the surface of the honeycomb carrier and at the same time increase the surface area of the carrier, thereby preparing the honeycomb carrier formed with a platinum coating layer with improved adhesion and physical properties such as corrosion resistance, durability, and wear resistance, and the cleaning solution includes a thiourea compound, hydrogen peroxide, amino acids, a chelating agent, a dispersion stabilizer, and deionized water.

Further, the cleaning solution includes 1 to 3% by weight of thiourea compound, 2 to 4% by weight of hydrogen peroxide, 1 to 3% by weight of amino acid, 0.1 to 0.3% by weight of chelating agent, 0.05 to 0.15% by weight of dispersion stabilizer, and the remaining amount of deionized water to satisfy 100% by weight.

The thiourea compound may be included to ensure that the cleaning solution remains stable when cleaning the honeycomb carrier, and the thiourea compound may include thiourea (NH₂)₂CS or a thiourea derivative represented by the general formula (R1R2N)(R3R4N)C═S (Here, R1, R2, R3, and R4 are each independently selected from a hydrogen atom, an ethyl group, or a methyl group).

The hydrogen peroxide is a substance used as an oxidizing agent, and the hydrogen peroxide can play a role in making cleaning easy.

The amino acid may act as a buffer against changes in acidity and as a sequestering agent for metal ions, and for example, the amino acid is an amino acid having 3 to 10 carbon atoms, and the amino acid may be one or more selected from the group consisting of isoleucine, arginine, proline, tyrosine, glutamic acid, glutamine, and glycine.

The chelating agent may be added to prevent a decrease in the cleaning ability of the cleaning solution and to prevent foreign substances or metal ions from being re-adsorbed through chemical bonds, and for example, the chelating agent may be diethylene trinitrilo pentaacetic acid (DTPA) and glutamic acid-2-acetic acid mixed at a weight ratio of 1:1.

The dispersion stabilizer may be used to stabilize the composition of the thiourea compound, hydrogen peroxide, amino acid, chelating agent, etc. and to protect the honeycomb carrier interface, and for example, the dispersion stabilizer may be a material represented by the following [Chemical Formula 1].

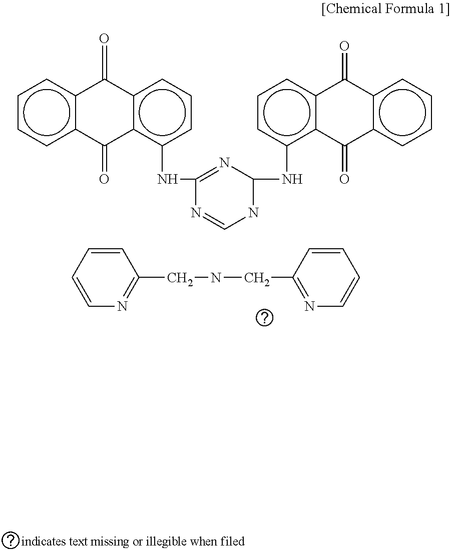

The deionized water is one in which ions have been removed from water, and it is desirable to use deionized water having a resistivity of 18 MΩ·cm or more.

### 3. S300: Platinum Catalyst Coating Solution Preparation Step

The platinum catalyst coating solution preparation step (S300) is a step of preparing a platinum catalyst coating solution to be coated on the honeycomb carrier.

In the platinum catalyst coating solution preparation step (S300), the platinum catalyst coating solution produced by the following method may be used.

To produce the platinum catalyst coating solution, first, materials consisting of deionized water, polyvinylpyrrolidone (PVP), platinum catalyst precursor, and alcohol may be prepared.

In the step, the materials may be prepared in a weight ratio of 30 to 50 parts by weight of deionized water, 20 to 30 parts by weight of polyvinylpyrrolidone (PVP), 1 to 10 parts by weight of platinum catalyst precursor, and 5 to 15 parts by weight of alcohol.

The polyvinylpyrrolidone (PVP) functions as a binder that binds the platinum halogen compound, and the polyvinylpyrrolidone is a water-soluble material and may be easily dissolved in water. The polyvinylpyrrolidone (PVP) is a polymer of N-vinyl-2-pyrrolidone, which is hygroscopic and is soluble in water and alcohol but insoluble in hydrocarbons. Those with low molecular weight are used as substitute plasma agents and are very stable substances in the human body. In addition, polyvinylpyrrolidone is used as a water-soluble adhesive, lipstick adhesive, and paper adhesive.

The platinum catalyst precursor may be a known platinum catalyst precursor for producing a platinum catalyst, and for example, known platinum catalyst precursors such as platinum chloride, platinum nitrate, K₂PtCl₆, and tetra-amine platinum nitrate (Pt(NH₃)₄(NO₃)₂) may be used as the platinum catalyst precursor.

Next, the deionized water, polyvinylpyrrolidone, platinum catalyst precursor, and alcohol can be mixed and then stirred to produce a mixed solution.

In the step, the mixed solution may be a uniformly mixed solution in which the deionized water, polyvinylpyrrolidone, platinum catalyst precursor, and alcohol are dissolved.

Next, the mixed solution may be heated and then cooled to produce a platinum catalyst coating solution.

In the step, the mixed solution is heated at a temperature of 80 to 90° C. for 10 to 30 minutes and then cooled at a temperature of 30 to 40° C. to produce a platinum catalyst coating solution in which the alcohol is vaporized and removed.

### 4. S400: Platinum Catalyst Coating Solution Applying Step

The platinum catalyst coating solution applying step (S400) is a step of coating the platinum catalyst coating solution on the honeycomb carrier.

In the platinum catalyst coating solution applying step (S400), the platinum catalyst coating solution may be coated on the honeycomb carrier using a known coating method, and for example, the honeycomb carrier may be coated by dipping it in the platinum catalyst coating solution, or the platinum catalyst coating solution may be sprayed on the honeycomb carrier.

The composition of the coating in the platinum catalyst coating solution applying step (S400) is a known technology, so detailed description thereof is excluded for convenience of explanation and clarity of the technical idea of the present invention.

### 5. S500: Drying Step

The drying step (S500) is a step of drying the honeycomb carrier coated with the platinum catalyst coating solution.

The drying step (S500) may be performed by drying the honeycomb carrier coated with the platinum catalyst coating solution at a temperature of 65 to 75° C. for 1 to 5 hours.

### 6. S600: Heat Treatment Step

The heat treatment step (S600) is a step of heat-treating the honeycomb carrier coated with the platinum catalyst coating solution.

In the heat treatment step (S600), the honeycomb carrier coated with the platinum catalyst coating solution is heat-treated to increase the number of micro-sized pores and increase the specific surface area of the platinum-coated honeycomb catalyst that is finally manufactured.

In the heat treatment step (S600), the honeycomb carrier coated with the platinum catalyst coating solution is heat treated at a temperature of 800 to 1,200° C. for 1 to 3 hours under an atmosphere of one or more inert gases selected from the group consisting of helium, nitrogen, and argon.

### 7. S700: Surface Treatment Step

Next, the surface treatment step (700) is a step of adding a reaction gas the heat-treated honeycomb carrier coated with platinum catalyst coating solution to form a nitride layer thereon, thereby producing a platinum-coated honeycomb catalyst.

In the surface treatment step (S700), a nitride layer may be formed by heating a reaction gas consisting of ammonia (NH3) to a certain temperature and then spraying it onto the heat-treated honeycomb carrier coated with the platinum catalyst coating solution for a certain period of time, and in the surface treatment step (S700), a nitride layer may be formed by heating a reaction gas consisting of ammonia (NH3) to 400 to 500° C. using a known reaction chamber and then spraying the heated reaction gas onto the top of the heat-treated honeycomb carrier coated with the platinum catalyst coating solution at an injection speed of 600 to 700 m/s,

At this time, the reaction gas consisting of ammonia (NH3) causes a reaction of 2NH₃⇔3H₂₊₂N, and as a result, nitrogen atoms cause a nitridation process on the surface of the honeycomb carrier coated with the platinum catalyst coating solution, forming a nitride layer with a thickness of 50 to 100 μm, thereby producing a platinum-coated honeycomb catalyst with excellent physical properties such as corrosion resistance, durability, and wear resistance.

Hereinafter, with reference to the attached drawings, a method for preparing platinum-coated honeycomb catalyst used in TOC water quality measuring device according to an embodiment of the technical idea of the present invention will be described in detail with embodiments. The following examples are intended to illustrate the present invention, but the present invention is not limited to the following examples and may be modified and changed in various ways.

### EXAMPLE

First, a honeycomb carrier made of alumina was prepared, and the prepared honeycomb carrier was cleaned with a cleaning solution to remove foreign substances attached to the surface of the honeycomb carrier.

At this time, the cleaning solution was prepared with 2% by weight of thiourea compound, 3% by weight of hydrogen peroxide, 2% by weight of amino acid, 0.2% by weight of chelating agent, 0.1% by weight of dispersion stabilizer, and the remaining amount of deionized water to satisfy 100% by weight.

Next, a platinum catalyst coating solution to be coated on the honeycomb carrier was prepared.

At this time, the platinum catalyst coating solution was prepared in a weight ratio of 40 parts by weight of deionized water, 25 parts by weight of polyvinylpyrrolidone (PVP), 5 parts by weight of a platinum catalyst precursor made of platinum chloride, and 10 parts by weight of alcohol, and the deionized water, polyvinylpyrrolidone, platinum catalyst precursor, and alcohol were mixed and then stirred to prepare a mixed solution, and then the mixed solution was heated at 85° C. for 20 minutes and then cooled at 35° C. to prepare a platinum catalyst coating solution in which the alcohol was vaporized and removed.

Next, the honeycomb carrier was immersed in the platinum catalyst coating solution to prepare a honeycomb carrier coated with the platinum catalyst coating solution, and the honeycomb carrier coated with the platinum catalyst coating solution was dried at a temperature of 70° C. for 3 hours.

Next, the dried honeycomb carrier coated with the platinum catalyst coating solution was heat-treated at 1,000° C. for 2 hours in an inert gas atmosphere consisting of argon.

Next, the heat-treated honeycomb carrier coated with platinum catalyst coating solution was added with a reaction gas to form a nitride layer thereon, thereby producing a platinum-coated honeycomb catalyst.

At this time, the nitride layer is formed by heating a reaction gas composed of ammonia (NH₃) to 450° C. using a reaction chamber, and then spraying the heated reaction gas onto the top of the heat-treated honeycomb carrier coated with platinum catalyst coating solution at an injection speed of 650 m/s.

### Comparative Example

First, a honeycomb carrier made of alumina was prepared,

Next, a platinum catalyst coating solution to be coated on the prepared honeycomb carrier was prepared.

At this time, the platinum catalyst coating solution was prepared in a weight ratio of 40 parts by weight of deionized water, 25 parts by weight of polyvinylpyrrolidone (PVP), 5 parts by weight of a platinum catalyst precursor made of platinum chloride, and 10 parts by weight of alcohol, and the deionized water, polyvinylpyrrolidone, platinum catalyst precursor, and alcohol were mixed and then stirred to prepare a mixed solution, and then the mixed solution was heated at 85° C. for 20 minutes and then cooled at 35° C. to prepare a platinum catalyst coating solution in which the alcohol was vaporized and removed.

Next, the honeycomb carrier was immersed in the platinum catalyst coating solution to prepare a honeycomb carrier coated with the platinum catalyst coating solution, and the honeycomb carrier coated with the platinum catalyst coating solution was dried at a temperature of 70° C. for 3 hours.

Next, the dried honeycomb carrier coated with the platinum catalyst coating solution was heat-treated at 1,000° C. for 2 hours in an inert gas atmosphere consisting of argon to produce platinum-coated honeycomb catalyst.

### Experimental Example 1

The stability, surface hardness, adhesion, and peelability of the platinum-coated honeycomb catalyst prepared according to Examples and Comparative Examples were measured in standard conditions, and the measurement results are shown in [Table 1] below.

TABLE 1

| | | Comparative |
|---|---|---|
| Classification | Example | Example |
| Stability | 1437 | 821 |
| Surface Hardness | Good | Weakness |
| Adhesion | Very Good | Middle |
| Peelability | No Peeling | Slight Peeling |

As shown in [Table 1], it can be confirmed that the platinum-coated honeycomb catalyst according to the Example not only has good surface hardness and adhesion, but also does not easily peel off from the surface and has excellent stability.

### Experimental Example 2

### (1) Surface Hardness Test

Pencil hardness was measured according to KS D 6711.

### (2) Water Resistance Test

The time for surface deformation (cracks, blisters, etc.) to occur continuously in hot water at 90° C. was measured.

The test results are shown in [Table 2].

TABLE 2

| | | Comparative |
|---|---|---|
| Classification | Example | Example |
| Surface Hardness | 3 H | 1 H |
| Water Resistance | 945 hr | 316 hr |

As shown in [Table 2] above, it can be confirmed that the platinum-coated honeycomb catalyst according to the Example has significantly excellent surface hardness and water resistance.

Although a preferred embodiment of the present invention has been described above with reference to the attached drawings, those skilled in the art to which the present invention pertains will understand that the present invention can be embodied in other specific forms without changing its technical spirit or essential features. Therefore, one embodiment described above should be understood as illustrative in all respects and not limiting.

1. A method for preparing a platinum-coated honeycomb catalyst used in toc water quality measuring device, the method comprising: a honeycomb carrier preparation step (S100) of preparing a honeycomb carrier to be coated with a platinum catalyst;
a honeycomb carrier cleaning step (S200) of cleaning the prepared honeycomb carrier with a cleaning solution;
a platinum catalyst coating solution preparation step (S300) of preparing a platinum catalyst coating solution to be coated on the honeycomb carrier;
a platinum catalyst coating solution applying step (S400) of coating the platinum catalyst coating solution on the honeycomb carrier;
a drying step (S500) of drying the honeycomb carrier coated with the platinum catalyst coating solution;
a heat treatment step (S600) of heat-treating the honeycomb carrier coated with the platinum catalyst coating solution; and
a surface treatment step (S700) of adding a reaction gas to the heat-treated honeycomb carrier coated with the platinum catalyst coating solution to form a nitride layer thereon, thereby producing the platinum-coated honeycomb catalyst.

2. The method of claim 1, wherein in the the platinum catalyst coating solution preparation step (S300), the platinum catalyst coating solution is produced by preparing materials consisting of deionized water, polyvinylpyrrolidone (PVP), platinum catalyst precursor, and alcohol, followed by mixing and then stirring the deionized water, polyvinylpyrrolidone (PVP), platinum catalyst precursor, and alcohol to produce a mixed solution, and followed by heating and then cooling the mixed solution.

3. The method of claim 2, wherein in the platinum catalyst coating solution preparation step (S300), the materials are prepared in a weight ratio of 30 to 50 parts by weight of deionized water, 20 to 30 parts by weight of polyvinylpyrrolidone (PVP), 1 to 10 parts by weight of platinum catalyst precursor, and 5 to 15 parts by weight of alcohol.

4. The method of claim 3, wherein in the platinum catalyst coating solution preparation step (S300), the mixed solution is heated at a temperature of 80 to 90° C. for 10 to 30 minutes and then cooled at a temperature of 30 to 40° C. to produce the platinum catalyst coating solution in which the alcohol is vaporized and removed.

5. The method of claim 4, wherein in the heat treatment step (S600), the honeycomb carrier coated with the platinum catalyst coating solution is heat-treated at a temperature of 800 to 1,200° C. for 1 to 3 hours under an atmosphere of at least one inert gas selected from the group consisting of helium, nitrogen and argon.
